(19) United States
(12) Patent Application Publication
(10) Pub. No.: US 2004/0137066 A1
(43) Pub. Date: Jul. 15, 2004

(54) Rationally designed therapeutic intravascular implant coating

(76) Inventor: Swaminathan Jayaraman, Fremont, CA (US)

Correspondence Address: PAUL D. BIANCO: FLEIT, KAIN, GIBBONS,, GUTMAN, BONGINI, & BIANCO P.L., 601 BRICKELL KEY DRIVE, SUITE 404, MIAMI, FL, 33131, US

(21) Appl. No.: 10/696,174

(22) Filed: Oct. 29, 2003

Related U.S. Application Data
(63) Continuation-in-part of application No. 09/994,253, filed on Nov. 26, 2001, now Pat. No. 6,641,611.
(63) Continuation-in-part of application No. 10/286,805, filed on Nov. 4, 2002.

(51) Int. Cl. A61K 9/14

(52) U.S. Cl. 424/486

(57) ABSTRACT

The invention relates to a method for treating a vascular disease of a patient. The method includes identifying a disease process in the pathology of a vascular disease, selecting a therapeutic agent to treat or prevent the vascular disease, coating an intravascular implant with a therapeutically effective amount of the agent, and implanting the intravascular implant in the patient. The invention also relates to a coating for an intravascular implant to treat or prevent a vascular disease of a patient. The coating includes a therapeutically effective amount of rapamycin and a therapeutically effective amount of a second agent. The second agent is selected based on the vascular disease of the patient.

### CROSS-REFERENCE TO RELATED APPLICATION

This application is a continuation-in-part of U.S. patent application Ser. No. 09/994,253, filed Nov. 26, 2001 and a continuation-in-part of U.S. patent application Ser. No. 10/286,805 filed Nov. 4, 2002.

### FIELD OF THE INVENTION

The present invention relates to a therapeutic coating for an intravascular implant, and in particular to a coating that prevents or treats vascular diseases.

### BACKGROUND OF THE INVENTION

As discussed in more detail below, the prior art discloses many examples of therapeutic coatings that have been applied to intravascular devices. The objective behind applying the therapeutic coating is to either mediate or suppress a tissue response at the site of implantation. For example in intravascular situations, one of the obvious outcomes of implanting a foreign body is for an intense reaction at the site of implantation. This intense reaction can result from either the implantation itself or the stresses generated after implantation. Due to the reaction, there is an obvious interaction by the vessel wall to compensate for this injury by producing a host of tissue related responses that is generally called “healing due to injury.” It is this healing process that the therapeutic coating attempts to mediate, suppress, or lessen. In some instances, this healing process is excessive in which it occludes the entire lumen providing for no blood flow in the vessel. This reoccluded vessel is also called a restenotic vessel.

Therapeutic coatings can behave in different ways. For example, depending upon the kind of therapeutic agent used, the various cellular levels of mechanisms are tackled. Some of the therapeutic agents act on the growth factors that are generated at the site of implantation or intervention of the vessel. Some other therapeutic agents act on the tissues and suppress the proliferative response of the tissues. Others act on the collagen matrix that comprises the bulk of the smooth muscle cells. Some examples of prior art relating to therapeutic coatings follow.

U.S. Pat. No. 5,283,257 issued to Gregory et al. provides a method of preventing or treating hyperproliferative vascular disease in a mammal by administering an amount of mycophenolic acid effective to inhibit intimal thickening. This drug can be delivered either after angioplasty or via a vascular stent that is impregnated with mycophenolic acid.

U.S. Pat. No. 5,288,711 issued to Mitchell et al. provides a method of preventing or treating hyperproliferative vascular disease in a mammal by administering an antiproliferative effective amount of a combination of rapamycin and heparin. This combination can be delivered either after angioplasty or via a vascular stent that is impregnated with the combination.

U.S. Pat. Nos. 5,516,781 and 5,646,160 issued to Morris et al. disclose a method of preventing or treating hyperproliferative vascular disease in a mammal by administering an antiproliferative effective amount of rapamycin alone or in combination with mycophenolic acid. The rapamycin or rapamycin/mycophenolic acid combination can be delivered via a vascular stent.

U.S. Pat. No. 5,519,042 issued to Morris et al. teaches a method of preventing or treating hyperproliferative vascular disease in a mammal consists of administering to a mammal an effective amount of carboxyamide compounds. This can also be delivered intravascularly via a vascular stent.

U.S. Pat. No. 5,646,160 issued to Morris et al. provides a method of preventing or treating hyperproliferative vascular disease in a mammal by administering an antiproliferative effective amount of rapamycin alone or in combination with mycophenolic acid. This can be delivered intravascularly via a vascular stent.

Each of the above-identified patents utilizes an immunosuppressive agent. Since the mid 1980's, many new small molecular weight molecules of natural product, semi-synthetic or totally synthetic origin have been identified and developed for the control of graft rejection. These include mizoribine, deoxyspergualin, cyclosporine, FK 506, mycophenolic acid (and its prodrug form as mycophenolate mofetil), rapamycin, and brequinar sodium. The mechanisms of some of these agents will now be briefly summarized.

Both cyclosporine and FK 506 suppress T-cell activation by impeding the transcription of selected cytokine genes in T cells. Neither has any known direct effects on B cells. The suppression of interleukin 2 (IL-2) synthesis is an especially important effect of these two agents, because this cytokine is required for T cells to progress from initial activation to DNA synthesis. Both cyclosporine A and FK 506 bind to cytoplasmic proteins. It has been recently proposed that cyclosporine A and FK 506 are bifunctional: one segment of the immunosuppressant molecule is responsible for binding to the rotamase and, once bound, a separate part of the molecule interacts with a cytoplasmic phosphatase (calcineurin) and causes the phosphatase to become inactive or have altered specificity. Unlike all previously developed immunosuppressants and even the most recent xenobiotic immunosuppressants, FK 506 is the only compound in the history of immunosuppressive drug development that is the product of a drug discovery program designed specifically to identify an improved molecule for the control of allograft rejection. Every other past and “new” immunosuppressive xenobiotic drug is the unanticipated result of drug discovery programs organized to identify lead compounds for anticancer, anti-inflammatory, or antibiotic therapy.

Neither cyclosporine, FK 506, rapamycin nor other immunosuppressants are the product of evolutionary pressures that led to our current use of them as immunosuppressants. The agents are fungal (cyclosporine A) or bacterial (FK 506, rapamycin) metabolites that suppress lymphocyte proliferation purely through coincidental molecular interactions. Therefore, as our ability to design drugs that perform specific intravascular functions increases, there should be a reciprocal decrease in the severity of their adverse effects.

There is a need for safer versions of cyclosporine, FK 506, rapamycin and mycophenolic acid as well as for analogues with higher immunosuppressive efficacy. Because of their toxicities, these agents cannot be used at maximally immunosuppressive doses.

Another significant issue that complicates the delivery of relatively high dosage of the agents is the relatively narrow therapeutic window. This narrow window of therapeutic vs. toxicity restricts most of these agents to be used as monotherapy for intravascular delivery.

Rapamycin, for example, inhibits the L-2 induced proliferation of specific IL-2 responsive cell lines, but neither cyclosporine nor other drugs can suppress this response. Because rapamycin acts late in the activation sequence of T cells, it also effectively inhibits T cells inactivated by a recently described calcium independent pathway. Thus, T cells stimulated through this alternative route are insensitive to suppression by cyclosporine A and FK 506, but rapamycin inhibits their proliferation only.

The toxicity profile of rapamycin resembles cyclosporine A and FK 506. Rapamycin is associated with weight loss in several species, and treatment with high doses of rapamycin causes diabetes in rats, but not in nonhuman primates. Initial animal data suggests that rapamycin may be less nephrotoxic than cyclosporine A, but its effects on kidneys with impaired function have not been evaluated. Rapamycin at highly effective therapeutic doses is highly toxic and its usage is recommended along with a combination of other immunosuppressants. The combination with cyclosporine A results in a significant increase in the therapeutic level in blood when compared with monotherapy. A lower dosage of the combination is more effective than a higher dosage of monotherapy. The dosage of rapamycin could be reduced nine fold and cyclosporine A could be reduced five fold when these agents are used in combination. In addition, the combination is also not toxic. In fact, the U.S. FDA has approved the usage of rapamycin for transplantation and allograft rejection only upon combination therapy with cyclosporine.

In summary, the problems associated with immunosuppressive agents include, narrow therapeutic window, toxicity window, inefficacy of agents, and dosage related toxicity. In order to overcome these problems, combination therapy involving two agents has been used with success. It has been surprisingly found that the benefits of combined immunosuppression with rapamycin and cyclosporine A have a very synergistic approach towards cellular growth and retardation. Studies have shown that suppression of heart graft rejection in nonhuman primates is especially effective when rapamycin is combined with cyclosporine A. The immunosuppressive efficacy of combined therapy is superior to treatment with either agent alone; this effect is not caused by the elevation of cyclosporine A blood levels by co-administration of rapamycin. The combination treatment with rapamycin and cyclosporine A does not cause nephrotoxicity. The distinct sites of immunosuppressive action of cyclosporine A and rapamycin (cyclosporine A acts on the calcium dependent and rapamycin acts on the calcium independent pathway) and their relatively non-overlapping toxicities will enable this combination to be used intravascularly to prevent cellular growth at the site of injury inside the blood vessel after angioplasty.

Several scientific and technical publications mention the “surprisingly” “synergistic” effect of rapamycin and cyclosporine A. These include:

Schuurman et al. in Transplantation Vol 64, 32-35, No. 1, Jul. 15, 1997 describe SDZ-RAD, a new rapamycin derivative that has a synergism with cyclosporine. They conclude that both the drugs show synergism in immunosuppression, both in vitro and in vivo. The drugs are proposed to have a promising combinatorial therapy in allotransplantation.

Schuler et al. in Transplantation Vol 64, 36-42, No. 1, Jul. 15, 1997 report that the drug rapamycin by itself has a very narrow therapeutic window, thus decreasing its clinical efficacy. They reported that in combination with cyclosporine A, the drugs act in a synergistic manner. This synergism, if proven in humans, offers the chance to increase the efficacy of the immunosuppressive regimen by combining the two drugs, with the prospect of mitigating their respective side effects. The authors also propose that they believe that the increased immunosuppressive efficacy of a drug combination composed of cyclosporine A and rapamycin, combined with the ability of rapamycin to prevent VSMC proliferation, bears the potential for improving the prospects for long term graft acceptance.

Morris et al. in Transplantation Proceedings, Vol 23, No. 1 (Feb), 1991: pp 521-524 describe the synergistic activity of cyclosporine A and rapamycin for the suppression of alloimmune reactions in vivo.

Schuurman et al. in Transplantation Vol 69, 737-742, No. 5, Mar. 15, 2000 describe the oral efficacy of the macrolide immunosuppressant rap amycin and of cyclosporine microemulsion in cynomalgus monkey kidney allotransplantation. The authors describe the synergistic activity of both these combinations and explain the possible explanation for failure of rapamycin monotherapy to ensure long term survival in this animal model might be the different mode of action of the compound when compared to cyclosporine. Cyclosporine acts very early in the chain of events that lead to a T-cell immune response. It blocks the antigen-driven activation of T cells, inhibiting the production of early lymphokines by interfering with the intracellular signal that emanates from the T-cell receptor upon recognition of antigen. Rapamycin acts rather late after T cell activation. The authors conclude that drugs like rapamycin need to be combined with immunosuppressants like cyclosporine to inhibit the early T-cell activation event and thus prevent an inflammatory response.

Hausen et al. in Transplantation Vol 69, 488-496, No. 4, Feb. 27, 2000 describe the prevention of acute allograft rejection in nonhuman primate lung transplant recipients. The authors mention that fixed dose studies using monotherapy with either high dose cyclosporine A or a high dose rapamycin did not prevent early acute allograft rejection, but monotherapy with either drug was well tolerated. The fixed doses of the drugs were used in combination, but this led to 5 fold increase in rapamycin levels compared to levels in monkeys treated with rapamycin alone. To compensate for this adverse drug-drug interaction, concentration controlled trials were designed to lower rapamycin levels and cyclosporine A levels considerably when both the drugs were used together. This specimen suppressed rejection successfully.

Martin et al. in the Journal of Immunology in 1995 published a paper “Synergistic Effect of Rapamycin and cyclosporine A in the Treatment of Experimental Autoimmune Uveoretinitis”. The authors conclude that immunosuppressive drugs currently available for the treatment of autoimmune diseases display a narrow therapeutic window between efficacy and toxic side effects. The use of combination of drugs that have a synergistic effect may expand this window and reduce the risk of toxicity. The studies demonstrated synergistic relationship between rapamycin and cyclosporine A and the combination allows the use of reduced doses of each drug to achieve a therapeutic effect. The use of lower doses may also reduce the toxicity of these drugs for the treatment of autoimmune uveitis.

Henderson et al. in Immunology 1991, 73: 316-321 compare the effects of rapamycin and cyclosporine A on the IL-2 production. While rapamycin did not have any effect on the IL-2 gene expression, cyclosporine A did have an effect on the IL-2 gene expression. This shows that the two drugs have a completely different pathway of action.

Hausen et al. in Transplantation Vol 67, 956-962, No. 7, Apr. 15, 1999 published the report of co administration of Neural (cyclosporine A) and the novel rapamycin analog (SDZ-RAD), to rat lung allograft recipients. They mention the synergistic effect of the two compounds—cyclosporine A inhibits early events after T-cell activation, rapamycin affects growth factor driven cell proliferation. Simultaneous administration of cyclosporine A and rapamycin has shown to result in significant increases in rapamycin trough (levels of the drug in blood) when compared with monotherapy. In preclinical and clinical trials, the immunosuppressive strategies have been designed to take advantage of the synergistic immunosuppressive activities of cyclosporine A given in combination with rapamycin. In addition to immunosuppressive synergism, a significant pharmacokinetic interaction after simultaneous, oral administration of cyclosporine A and rapamycin has been found in animal studies.

Whiting et al. in Transplantation Vol 52, 203-208, No. 2, August 1991 describe the toxicity of rapamycin in a comparative and combination study with cyclosporine at immunotherapeutic dosage in the rat.

Yizheng Tu et al. in Transplantation Vol 59, 177-183, No. 2 Jan. 27, 1995 published a paper on the synergistic effects of cyclosporine, Siolimus (rapamycin) and Brequinar on heart allograft survival in mice.

Yakimets et al. in Transplantation Vol 56, 1293-1298, No. 6, December 1993 published the “Prolongation of Canine Pancreatic Islet Allograft Survival with Combined rapamycin and cyclosporine Therapy at Low Doses”.

Vathsala et al. in Transplantation Vol 49, 463-472, No. 2, February 1990 published the “Analysis of the interactions of Immunosuppressive drugs with cyclosporine in inhibiting DNA proliferation”.

The combination of rapamycin and cyclosporine A, delivered by a variety of mechanisms, has been patented for the treatment of many diseases. The patent literature is summarized below:

U.S. Pat. No. 5,100,899 issued to Calne provides a method of inhibiting organ or tissue transplant rejection in a mammal. The method includes administering to the mammal a transplant rejection inhibiting amount of rapamycin. Also disclosed is a method of inhibiting organ or tissue transplant rejection in a mammal that includes administering (a) an amount of rapamycin in combination with (b) an amount of one or more other chemotherapeutic agents for inhibiting transplant rejection, e.g., azathiprine, corticosteroids, cyclosporine and FK 506. The amounts of (a) and (b) together are effective to inhibit transplant rejection and to maintain inhibition of transplant rejection.

U.S. Pat. No. 5,212,155 issued to Calne et al. Claims a combination of rapamycin and cyclosporine that is effective to inhibit transplant rejection.

U.S. Pat. No. 5,308,847 issued to Calne describes a combination of rapamycin and axathioprine to inhibit transplant rejection.

U.S. Pat. No. 5,403,833 issued to Calne et al. described a combination of rapamycin and a corticosteroid to inhibit transplant rejection.

U.S. Pat. No. 5,461,058 issued to Calne describes a combination of rapamycin and FK 506 to inhibit transplant rejection.

Published U.S. Patent Application No. US2001/0008888 describes a synergistic combination of IL-2 transcription inhibitor (e.g., cyclosporine A) and a derivative of rapamycin, which is useful in the treatment and prevention of transplant rejection and also certain autoimmune and inflammatory diseases, together with novel pharmaceutical compositions comprising an IL-2 transcription inhibitor in combination with rapamycin.

U.S. Pat. No. 6,239,124 issued to Zenke et al. also describes a synergistic combination of IL-2 transcription inhibitor and rapamycin which is useful in the treatment and prevention of transplant rejection and also certain autoimmune and inflammatory diseases, together with novel pharmaceutical compositions comprising an IL-2 transcription inhibitor in combination with rapamycin.

U.S. Pat. No. 6,051,596 issued to Badger describes a pharmaceutical composition containing a non-specific suppressor cell inducing compound and cyclosporine A in a pharmaceutically acceptable carrier. The patent also discloses a method of inducing an immunosuppressive effect in a mammal, which comprises administering an effective dose of the non-specific suppressor cell inducing compound and cyclosporine A to such mammal.

U.S. Pat. No. 6,046,328 issued to Schonharting et al. describes the preparation and combination of a Xanthine along with cyclosporine A or FK 506.

U.S. Pat. Nos. 5,286,730 and 5,286,731 issued to Caufield et al. describe the combination of rapamycin and cyclosporine A useful for treating skin diseases, and the delivery of the above compounds orally, parentally, intranasally, intrabronchially, topically, transdermally, or rectally.

Published International Application No. WO 98/18468 describes the synergistic composition comprising rapamycin and Calcitriol.

U.S. Pat. Nos. 5,624,946 and 5,688,824 issued to Williams et al. describe the use of Leflunomide to control and reverse chronic allograft rejection.

U.S. Pat. No. 5,496,832 issued to Armstrong et al. provides a method of treating cardiac inflammatory disease which comprises administering rapamycin orally, parenterally, intravascularly, intranasally, intrabronchially, transdermally or rectally.

The concept of restenosis or hyperproliferative vascular disease is now being more clearly understood then it was a couple of years ago. The distinctive feature of restenosis is its diverse histopathology. Histologically, restenosis is characterized by a diffuses, concentric, fibrous expansion of the graft arterial intima, termed neointimal hyperplasia. Growth of this lesion, which is often accompanied by fragmentation of the internal elastic lamina, results in progressive vascular occlusion and is seen as a reduction in lumen cross-sectional area in histological sections or upon angiography or other intravascular techniques. Neointimal hyperplasia, together with constrictive vascular remodeling, eventually culminates in complete arterial occlusion.

Restenosis was simply thought to be a response of the vascular smooth muscle cells upon injury. There is now information available to demonstrate that the restenosis process is different in every individual depending on the underlying conditions that constitute the vascular disease. These underlying conditions can be classified as diabetic, non-diabetic, small vessels, larger vessels, complex diseases, pro-atherogenic vessels, etc. Depending on the various mechanisms of the underlying complications, the restenotic process is different and various drugs and combination of drugs can used to treat or prevent a specific disease process of the vascular disease.

### SUMMARY OF THE INVENTION

In accordance with one aspect of the present invention, a method for treating a vascular disease of a patient with an intravascular implant is provided. The method includes identifying a disease process in the pathology of the vascular disease and selecting a first agent to treat or prevent the vascular disease. The method also includes coating at least a portion of the intravascular implant with a therapeutically effective amount of the first agent and implanting the intravascular implant in the patient.

In accordance with another aspect of the present invention, a method of making a therapeutically coated intravascular implant for treating a vascular disease is provided. The method includes identifying a disease process in the pathology of a vascular disease of a patient and selecting a first agent to treat or prevent the vascular disease. The method also includes coating at least a portion of the intravascular implant with a therapeutically effective amount of the first agent.

The disease process may be identified using an angiogram, fluoroscopy, CT scan, MRI, intravascular MRI, lesion temperature determination, genetic determination, or combination thereof. The disease process may include acute myocardial infarction, thrombotic lesions, unstable angina, fibrotic disease, total occlusion, hyperproliferative vascular disease, vulnerable plaque, diabetic vascular diffused disease, or a combination thereof. The first agent may act on a calcium independent cellular pathway or may be a macrolide immunosuppressant, like rapamycin.

The method may further include selecting a second agent to treat or prevent the vascular disease and coating at least a portion of the intravascular implant with a therapeutically effective amount of the second agent. The second agent may be an anti-inflammatory agent, non-proliferative agent, anti-coagulant, anti-platelet agent, Tyrosine Kinase inhibitor, anti-infective agent, anti-tumor agent, anti-leukemic agent, or a combination thereof.

Moreover, the method may include coating at least a portion of the intravascular implant with a polymer matrix. The polymer matrix may include a bioabsorbable polymer which can include poly-α hydroxy acids, polyglycols, polytyrosine carbonates, starch, gelatins, cellulose and combinations thereof. The therapeutically effective amount of the first agent may be dispersed within the bioabsorbable polymer.

Furthermore, the intravascular implant may be, but is not limited to, a balloon catheter, stent, stent graft, drug delivery catheter, atherectomy device, filter, scaffolding device, anastomotic clip, anastomotic bridge, suture material, metallic or non-metallic wire, embolic coil or a combination thereof. The intravascular implant may include a primer layer upon which the coating is applied. The primer layer may be made of a bioabsorbable polymer or a biostable polymer. Also, the coating may include a top coat applied over the coating.

In accordance with still another aspect of the present invention, a therapeutic intravascular implant coating for coating at least a portion of an intravascular implant to treat or prevent a vascular disease of a patient is provided. The coating includes a therapeutically effective amount of rapamycin and a therapeutically effective amount of a second agent. The second agent is selected based on the vascular disease of the patient.

The second agent may be an anti-inflammatory agent, non-proliferative agent, anti-coagulant, anti-platelet agent, Tyrosine Kinase inhibitor, anti-infective agent, anti-tumor agent, anti-leukemic agent, or a combination thereof.

A more detailed explanation of the invention is provided in the following description and claims, and is illustrated in the accompanying drawings.

### BRIEF DESCRIPTION OF THE DRAWINGS

Preferred features of the present invention are disclosed in the accompanying drawings, wherein similar reference characters denote similar elements throughout the several views, and wherein:

FIG. 1 shows the chemical structures of various macrocyclic immunosuppressants;

FIG. 2 shows a schematic of possible sites of action of cyclosporine A, FK 506, rapamycin, mizoribine, mycophenolic acid, brequinar sodium, and deoxyspergualin on T cell activation by calcium dependent or independent pathways. Certain immunosuppressants also affect B cells and their possible sites of action are also shown;

FIG. 3 shows a schematic of the effects of cyclosporine A, FK 506, rapamycin, mizoribine, mycophenolic acid, and brequinar sodium on the biochemistry of T cell activation;

FIG. 4 shows a graph comparing the effects of cyclosporine A alone (white bars), rapamycin alone (hatched bars), and the combination of cyclosporine A and rapamycin (black bars) on the proliferative response of cells;

FIG. 5 shows an isobologram analysis of a combination of cyclosporine A and rapamycin. The line drawn from 1 to 1 is the line of unity. Combinations that fall below this unity line are synergistic, on the line additive, and above the line antagonistic. The units on the X-axis are Fractional Inhibitory Concentration (FIC) of rapamycin and the units on the Y-axis are FIC of cyclosporine A;

FIG. 6 shows an isobologram analysis of a combination of cyclosporine A and rapamycin. The units on the X-axis are FIC of rapamycin and the units on the Y-axis are FIC of cyclosporine A. The combination at which the maximum proliferative response was inhibited was used to plot the synergistic interaction between the two;

FIG. 7 shows a graph illustrating the amount of proliferation of vascular smooth cells based on different treatments.

FIG. 8 shows a rationally designed, therapeutically coated stent preform;

FIG. 9 shows a cross-sectional view through one embodiment of the stent preform;

FIG. 10 shows cross-sectional view through another embodiment of the stent preform;

FIG. 11 shows yet another embodiment of the stent preform including a lubricious lining;

FIG. 12 shows still another embodiment of the stent preform using a tape as an outer sheathing; and

FIG. 13 shows a braided stent formed from a stent preform.

### DETAILED DESCRIPTION OF THE INVENTION

In the description that follows, any reference to either orientation or direction is intended primarily for the convenience of description and is not intended in any way to limit the scope of the present invention thereto. Further, any reference to a particular biological application or implant, such as use of a stent for cardiovascular applications, is simply used for convenience as one example of a possible use for the invention and is not intended to limit the scope of the present invention thereto.

According to the present invention, a coating for an intravascular implant is provided. The coating can be applied either alone, or within a polymeric matrix, which can be biostable or bioabsorbable, to the surface of an intravascular device. The coating can be applied directly to the implant or on top of a polymeric substrate, i.e. a primer. If desired, a top coat can be applied to the therapeutic coating.

The intravascular implant coating according to the present invention comprises a therapeutically effective amount of a first agent, the first agent acting on a calcium independent cellular pathway, and a therapeutically effective amount of a second agent, the second agent acting on a calcium dependent cellular pathway. The combined amount of the first and second agents treats or prevents hyperproliferative vascular disease. In an exemplary embodiment, the first agent is rapamycin and the second agent is cyclosporine A.

Alternatively, the therapeutic intravascular implant coating includes an effective amount of at least one therapeutic agent to treat or prevent a disease process of a vascular disease of a patient, wherein the effective amount of at least one therapeutic agent cures the vascular disease.

Also, the intravascular implant coating according to the present invention includes a therapeutically effective amount of a first agent to treat or prevent a disease process of a vascular disease of a patient. The first agent acts on a calcium independent cellular pathway and may be a macrolide immunosuppressant, or more specifically, rapamycin. The intravascular implant coating also includes a therapeutically effective amount of a second agent to treat or prevent the disease process of the vascular disease of the patient. The second agent may be an anti-inflammatory agent, non-proliferative agent, anti-coagulant, anti-platelet agent, Tyrosine Kinase inhibitor, anti-infective agent, anti-tumor agent, anti-leukemic agent, or a combination thereof.

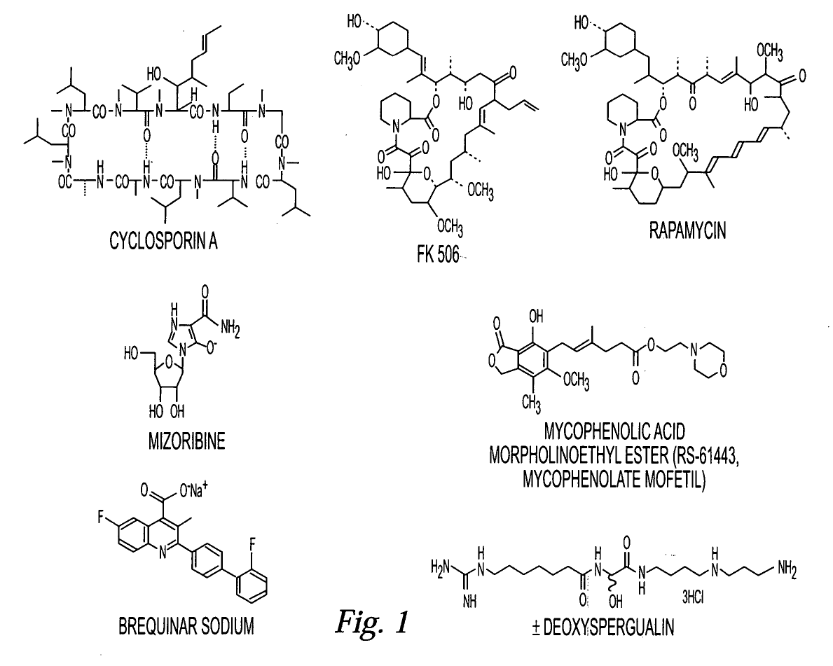

FIG. 1 shows some therapeutic agents and chemical structures used in the present invention. The distinct sites of action of rapamycin, which is a macrolide immunosuppressant acting on a calcium independent pathway, and cyclosporine A, which is an IL-2 transcription inhibitor acting on a calcium dependent pathway, and their relatively non-overlapping toxicities will enable this combination to be used intravascularly after angioplasty to prevent cellular growth at the site of injury inside the vessel.

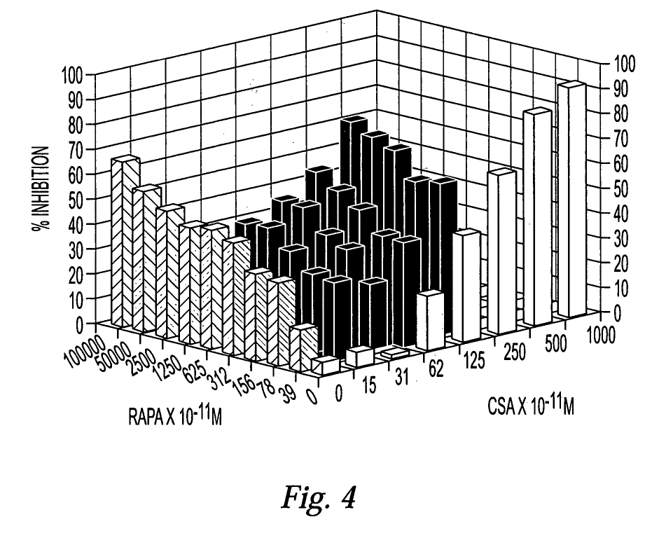

The rationale for a combinatorial therapy for intravascular therapy is at least in part as follows. The immunosuppressive efficacy to prevent allograft rejection after staggered administration of the two agents was similar to that obtained with simultaneous administration of combined therapy and significantly reduced the incidence of rejection in cardiac allografts (FIG. 4).

In the past, clinicians have learned to take advantage of known interactions between cyclosporine A and other compounds such as “azole” antifungals to reduce cyclosporine A dose requirements. In particular, the azole antifungals have no known clinically significant immunosuppressive properties and have little toxicity at the doses used in this context. Because in this context, they are not given for their pharmocodynamic effects. The amount of absorption of the azole antifungals is not critical. In the case of co-administration of cyclosporine A and rapamycin, both agents have low and variable bioavailabilities as well as narrow therapeutic indices. In addition, this interaction is dose dependent and can be completely avoided with low doses of combinatorial delivery.

In some aspects, the process of allograft rejection is similar to the restenosis process inside the coronary arteries after injury to the vessel wall. After arterial injury, multiple mitogenic and proliferative factors have been identified as capable of triggering signaling mechanisms leading to SMC activation. Because rapamycin and cyclosporine combination targets fundamental regulators of cell growth, it may significantly reduce restenosis.

A coating for an intravascular implant that includes the combination of rapamycin and cyclosporine A helps ensure that the mediation of cell growth happens very early in the cell cycle. For example, cyclosporine A acts early after T cell activation, thereby blocking transcriptional activation of early T cell specific genes. Rapamycin acts later in the cell cycle by blocking growth factor driven cell proliferation. The two agents can be provided in the coating such that the amount of rapamycin is higher than cyclosporine A. Thus, the ratio of rapamycin to cyclosporine A could be about 51% and above.

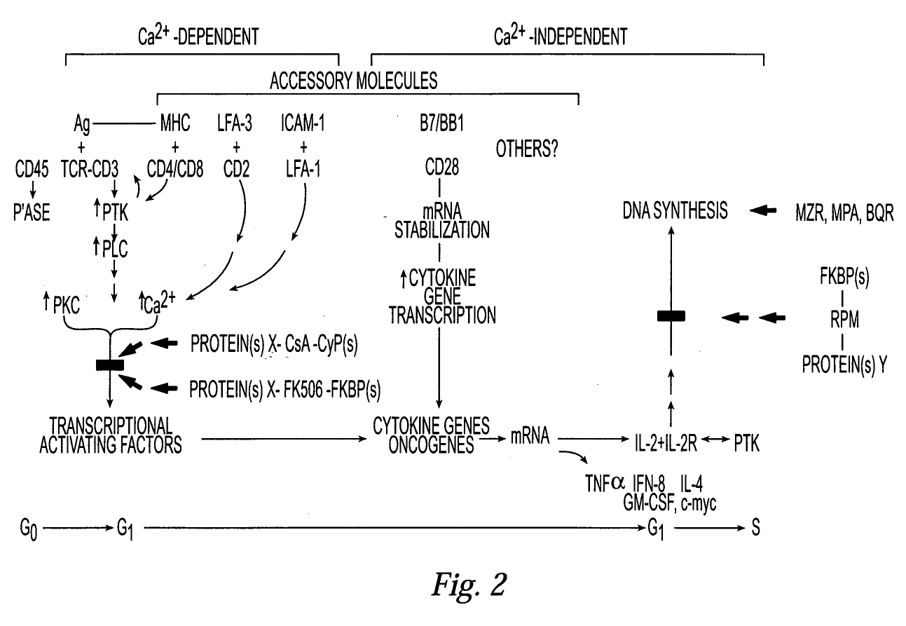

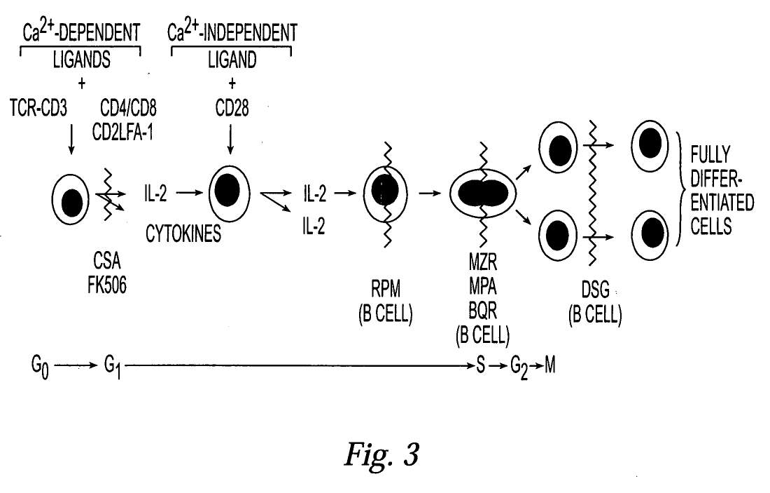

As shown in FIGS. 2 and 3, the activation of T cells, which seems to be critical for induction of host resistance and consequent rejection of the transplanted organ, occurs in three phases. The first phase causes transcriptional activation of immediate and early genes (IL-2 receptor) that allow T cells to progress from a quiescent (G0) to a competent (G1) state. In the second phase, T cells transduce the signal triggered by stimulating cytokines in both an autocrine and a paracrine fashion permitting entry into the cell cycle with resultant clonal expansion and acquisition of effector functions in the third phase of the immune response. Cyclosporine A inhibits the first phase and rapamycin inhibits the second phase of T cell activation. By ensuring that the stent surface or any intravascular surface has both these drugs, it is ensured that the restenotic response from the arterial wall is significantly reduced or is completely eliminated.

Although the two agents could be used separately, a considerable over dosing has to be done to ensure that both the agents have a necessary therapeutic effect. This overdosing could potentially result in side effects, which include improper healing of the vessel and also an incomplete intimal formation.

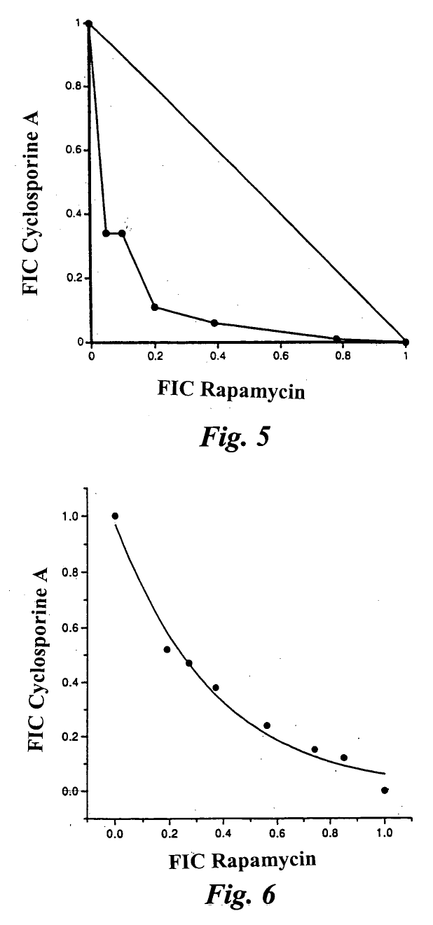

The combination of the agents would mean that both agents can be combined at a very low dosage and the combination would actually increase the therapeutic levels rather than administering monotherapy. This is illustrated in FIGS. 5 and 6, which shows the synergistic effects of rapamycin and cyclosporine A. The toxicity of the combination of agents is significantly reduced when both are combined together. Providing two agents that are active on two different cell cycles to prevent proliferation increases the therapeutic window of the agent. The combination actually increases the level of immunosuppression when compared to monotherapy.

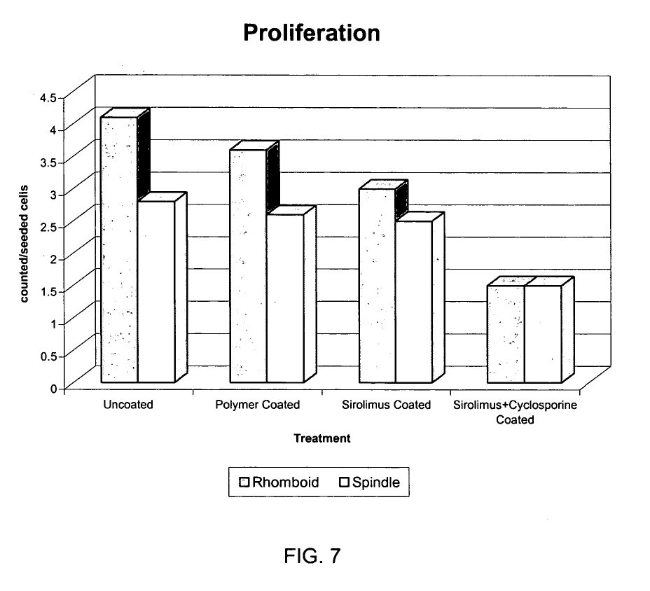

FIG. 7 illustrates the amount of proliferation of vascular smooth muscle cells based on different treatments. The vascular wall primarily consists of smooth muscle cells. The proliferation of these smooth muscle cells cause hyperproliferative vascular disease or restenosis. There are generally two types of smooth muscle cells: rhomboid shaped and spindle shaped. Rhomboid shaped cells are seen in a normal vessel wall, while spindle shaped cells are seen during restenosis (after balloon angioplasty or stenting). The graph of FIG. 7 shows that combinatorial therapy is more effective in preventing both types of smooth muscle cells than monotherapy. The graph shows an amount of proliferation which is less with rapamycin and cyclosporine (combinatorial therapy) than with rapamycin alone (monotherapy). Uncoated and polymer coated implants show greater amounts of proliferation when compared to combinatorial therapy.

In another embodiment of the present invention, a therapeutic intravascular implant coating to treat or prevent a disease process of a vascular disease is described. That is, a coating based on the genesis of the disease and the underlying morphology of the disease. This concept of a therapeutic coating evolved from the need to identify key events in the molecular pathology of fibroproliferative restenotic disease in order to develop specific and effective treatments. Restenosis is no longer just identified as a hyperproliferative disease, but more specifically it is viewed as a fibroproliferative disease with well defined pathologic cascade of events and interactions.

Therefore, therapeutic agents to be delivered via an implant coating or stent into the vascular vessel wall are designed to treat or prevent the disease processes that create the problem. The disease processes can include, but are not limited to, acute myocardial infarction, thrombotic lesions, unstable angina, fibrotic disease, total occlusion, hyperproliferative vascular disease, vulnerable plaque, and diabetic vascular diffused disease.

Techniques used to identify these events or processes include an angiogram, fluoroscopy, CT scan, MRI, intravascular MRI, lesion temperature determination, genetic determination, etc. An angiogram is acquired by injecting a radiopaque dye into the vascular system, usually by means of a catheter. The radiopaque dye infuses the vessels, and a radiological projection is made of the infused vessels onto a radiographic sensor. The resultant angiogram will reveal the lumens of the vessels as the radiopaque dye flows through them. A narrowing of the infused lumen will provide an indication of an obstruction of a vessel and a potential condition for infarction.

Fluoroscopy generates images of internal structures on a video monitor during energization of an x-ray tube. Fluoroscopy may use x-ray to produce real-time video images. After the x-rays pass through the patient, they are captured by a device called an image intensifier and converted into light. The light is then captured by a TV camera and displayed on a video monitor.

A CT scan (computed tomography scan) is a special radiographic technique that uses a computer to assimilate multiple X-ray images into a 2 dimensional cross-sectional image. This can reveal many soft tissue structures not shown by conventional radiography. Scans may also be dynamic in which a movement of a dye is tracked. A special dye material may be injected into the patient's vessel prior to the scan to help differentiate abnormal tissue and the vasculature.

An MRI scan (magnetic resonance imaging scan) is a method of visualizing soft tissues of the body by applying an external magnetic field that makes it possible to distinguish between hydrogen atoms in different environments. The images are very clear and are particularly good for the brain, spinal cord, joints, abdomen and soft tissue. Intravascular MRI uses an MR probe which may be built into catheters, allowing diagnostically useful high resolution images to be obtained from within small, intravascular structures.

Identifying lesion temperature is a technique without significant clinical experience. The temperature of a lesion is measured to determine whether it is unstable or not. A catheter, probe, or the like, is inserted into the vasculature near the lesion, and the temperature of the lesion may be measured. For example, one technique is measuring lesion temperature by analyzing stress patterns in a lesion molding balloon which are revealed under a polariscope after the balloon has been molded to the lesion and then removed from the body for inspection. In another example, a balloon coating which changes color in accordance with a temperature experience may be used. Also, temperature of lesion may be measured using an infrared sensor.

Finally, genetic determination is a technique to identify differently expressed genes in the process of a vascular disease. The systematic and comprehensive characterization of gene transcription is possible using whole genome sequencing, bioinformatics and high throughput transcription profiling technologies. Based on specifically identified genes in a vascular disease, a disease process can be identified, and the vascular disease may be treated or prevented.

Given the identification of all the different processes of restenosis, construction of a disease specific therapeutic coatings can be designed which can be used to treat or prevent processes of restenosis from people with various risk factors and underlying mechanisms. That is, restenosis is different in every individual depending on the underlying conditions that constitute the vascular disease.

To make a therapeutic intravascular implant coating to treat or prevent a specific disease process of a vascular disease, the disease process or processes which are prevalent in the vessel wall of the patient are identified. This identification can be achieved using a technique or a combination of techniques previously mentioned. A therapeutic agent or combination of agents is selected for treating or preventing the identified disease process or processes. The intravascular implant coating includes a therapeutically effective amount of a first agent to treat or prevent the disease process.

One way to identify a disease process in the vessel wall of the patient and to treat the vascular disease is to perform more than one procedure on the patient. First, a preliminary procedure may be performed with the goal of determining the prevalent disease process. Based on the identification of the disease process, an implant may be coated with at least one therapeutic agent, or a pre-coated implant having the desired therapeutic agent or agents may be obtained. Then, the patient may undergo another procedure for implanting the coated implant in the patient's vasculature. Alternatively, a single procedure may be performed to identify the disease process and insert the coated implant in the patient. In this regard, it is envisioned that the implant could be coated with the desired agent or agents at the site of the procedure, or a pre-coated implant having the desired therapeutic agent or agents may be selected from an inventory of pre-coated implants.

In one embodiment, the first agent acts on a calcium independent cellular pathway and may be a macrolide immunosuppressant, or more specifically, rapamycin. The intravascular implant coating also includes a therapeutically effective amount of a second agent to treat or prevent the disease process of the vascular disease. The second agent can include an anti-inflammatory agent, non-proliferative agent, anti-coagulant, anti-platelet agent, Tyrosine Kinase inhibitor, anti-infective agent, anti-tumor agent, anti-leukemic agent, or any combination thereof.

Examples of anti-inflammatory agents include, but are not limited to, Zinc compounds, dexamethasone and its derivatives, aspirin, non-steroidal anti-inflammatory drugs (NSAIDs) (such as ibuprofen and naproxin), TNF-α inhibitors (such as tenidap and rapamycin or derivatives thereof), or TNF-α antagonists (e.g., infliximab, OR1384), prednisone, dexamethasone, Enbrel®, cyclooxygenase inhibitors (i.e., COX-1 and/or COX-2 inhibitors such as Naproxen®, Celebrex®, or Vioxx®), CTLA4-Ig agonists/antagonists, CD40 ligand antagonists, other IMPDH inhibitors, such as mycophenolate (CellCept®), integrin antagonists, alpha-4 beta-7 integrin antagonists, cell adhesion inhibitors, interferon gamma antagonists, ICAM-1, prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, therapies for the treatment of irritable bowel syndrome (e.g., Zelmac® and Maxi-K® openers), or other NF-κB inhibitors, such as corticosteroids, calphostin, CSAIDs, 4-substituted imidazo [1,2-A]quinoxalines, glucocorticoids, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Examples of anti-proliferative agents include, but are not limited to, cytochalasins, Taxol®, somatostatin, somatostatin analogs, N-ethylmaleimide, antisense oligonucleotides and the like, cytochalasin B, staurosporin, nucleotide analogs like purines and pyrimidines, Taxol®, topoisomerase inhibitor like topoisomerase I inhibitor or a topoisomerase II inhibitor, alkylating agents such as nitrogen mustards (mechlorethamine, cyclophosphamide, melphalan (L-sarcolysin)), nitrosoureas (carmustine (BCNU), lomustine (CCNU), semustine (methyl-CCNU), streptozocin, chlorozotocin), immunosuppressants (mycophenolic acid, thalidomide, desoxyspergualin, azasporine, leflunomide, mizoribine, azaspirane (SKF 105685)), paclitaxel, altretamine, busulfan, chlorambucil, cyclophosphamide, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, prednisone, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea.

Examples of anti-coagulant agents include, but are not limited to, an RGD peptide-containing compound, heparin, antithrombin compounds, platelet receptor antagonists, anti-thrombin antibodies, anti-platelet receptor antibodies, aspirin, protaglandin inhibitors, platelet inhibitors, tick anti-platelet peptide, hirudin, hirulog, and warfarin.

Examples of anti-platelet agents include, but are not limited to, ReoPro®, ticlopidine, clopidrogel, and fibrinogen receptor antagonists.

Examples of Tyrosine Kinase inhibitors include, but are not limited to, c-Met, a receptor tyrosine kinase, and its ligand, scatter factor (SF), Epithelial Cell Kinase (ECK), inhibitors described in international patent applications WO 96/09294 and WO 98/13350 and U.S. Pat. No. 5,480,883 to Spada, et al., certain 2,3-dihydro-1H-[1,4]oxazino[3,2-g]quinolines, 3,4-dihydro-2H-[1,4]oxazino[2,3-g]quinolines, 2,3-dihydro-1H-[1,4]thiazino[3,2-g]quinolines, and 3,4-dihydro-2H-[1,4]thiazino[2,3-g]quinolines, EGF, PDGF, FGF, src tyrosine kinases, PYK2 (a newly discovered protein tyrosine kinase) and PTK-X (an undefined protein tyrosine kinase).

Examples of anti-infective agents include, but are not limited to Leucovorin, Zinc compounds, cyclosporins (e.g., cyclosporin A), CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, monoclonal antibody OKT3, agents blocking the interaction between CD40 and CD154 (a.k.a. “gp39”), such as antibodies specific for CD40 and/or CD154, fusion proteins constructed from CD40 and/or CD154/gp39 (e.g., CD40Ig and CD8gp39), β-lactams (e.g., penicillins, cephalosporins and carbopenams), β-lactam and lactamase inhibitors (e.g., augamentin), aminoglycosides (e.g., tobramycin and streptomycin), macrolides (e.g., erythromycin and azithromycin), quinolones (e.g., cipro and tequin), peptides and deptopeptides (e.g. vancomycin, synercid and daptomycin), metabolite-based anti-biotics (e.g., sulfonamides and trimethoprim), polyring systems (e.g., tetracyclins and rifampins), protein synthesis inhibitors (e.g., zyvox, chlorophenicol, clindamycin, etc.), nitro-class antibiotics (e.g., nitrofurans and nitroimidazoles), fungal cell wall inhibitors (e.g., candidas), azoles (e.g., fluoconazole and vericonazole), membrane disruptors (e.g., amphotericin B), nucleoside-based inhibitors, protease-based inhibitors, viral-assembly inhibitors, and other antiviral agents such as abacavir.

Examples of anti-tumor agents include, but are not limited to, DR3 Ligand (TNF-Gamma) and MIBG.

Examples of anti-leukemic agents include, but are not limited to, mda-7, human fibroblast interferon, mezerein, and Narcissus alkaloid (pretazettine).

Examples of chemotherapeutic agents include, but are not limited to, antibiotic derivatives (e.g., doxorubicin, bleomycin, daunorubicin, and dactinomycin), antiestrogens (e.g., tamoxifen), antimetabolites (e.g., fluorouracil, 5-FU, methotrexate, floxuridine, interferon alpha-2b, glutamic acid, plicamycin, mercaptopurine, and 6-thioguanine), cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate), hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone), nitrogen mustard derivatives (e.g., mephalen, chorambucil, mechlorethamine (nitrogen mustard) and thiotepa), steroids and combinations (e.g., bethamethasone sodium phosphate), and others (e.g., dicarbazine, asparaginase, mitotane, vincristine sulfate, vinblastine sulfate, and etoposide).

Examples of anti-angiogenic inhibitors include, but are not limited to, AG-3340 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), Metastat (Aeterna, St-Foy, Quebec), EMD-121974 (Merck KcgaA Darmstadt, Germany), Vitaxin (Ixsys, La Jolla, Calif./Medimmune, Gaithersburg, Md.), Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), SU-6668 (Sugen), IM-862 (Cytran, Kirkland, Wash.), Interferon-alpha, IL-12 (Roche, Nutley, N.J.), and Pentosan polysulfate (Georgetown University, Washington, D.C.).

Other therapeutic agents include thrombolytic agents such as tissue plasminogen activator, streptokinase, and urokinase plasminogen activators; lipid lowering agents such as antihypercholesterolemics (e.g. HMG CoA reductase inhibitors such as mevastatin, lovastatin, simvastatin, pravastatin, and fluvastatin, HMG CoA synthatase inhibitors, etc.); and anti-diabetic drugs, or other cardiovascular agents (loop diuretics, thiazide type diuretics, nitrates, aldosterone antagonistics (i.e. spironolactone and epoxymexlerenone), angiotensin converting enzyme (e.g. ACE) inhibitors, angiotensin II receptor antagonists, beta-blockers, antiarrythmics, anti-hypertension agents, and calcium channel blockers).

In one example of combinatorial therapy, rapamycin may be combined with Gleevec®. Gleevec® is a compound which is highly selective for PDGFR alpha, Beta-associated v-Abl tyrosine kinase. These compounds are not only able to inhibit acute vascular lesion formation after denudation injury, but also the development of chronic lesions such as those seen in diffused diseases in the vessel wall. Gleevec® can be combined with rapamycin and delivered to the vessel wall via an intravascular implant.

As another example, heparin is known to dissolve clots in the vessel wall. By combining heparin with rapamycin, the stent is much less susceptible to clot formation.

In still another example, curcumin (diferuloylmethane), an anti-inflammatory agent from curcuma longa, affects the proliferation of blood mononuclear cells and vascular smooth muscle cells. Curcumin independently inhibited the proliferation of rabbit vascular smooth muscle cells stimulated by fetal calf serum. Curcumin had a greater inhibitory effect on platelet derived growth factor stimulated proliferation than on serum-stimulated proliferation. Curcumin is very useful in the prevention of pathologic changes of atherosclerosis and restenosis. The possible mechanisms of the antiproliferative and apoptic effects of curcumin on vascular smooth muscle cells were studied in rat aortic smooth muscle cell line. Curcumin inhibits cell proliferation, arrested the cell cycle progression and induced cell apoptosis in vascular smooth muscle cells.

It should be noted that the present invention relates to a combinatorial therapy for delivery of more than one agent through a coating on any intravascular implant. As used herein, implant means any type of medical or surgical implement, whether temporary or permanent. Delivery can be either during or after an interventional procedure. The intravascular implant may be, but is not limited to, a balloon catheter, stent, stent graft, drug delivery catheter, atherectomy device, filter, scaffolding device, anastomotic clip, anastomotic bridge, suture material, metallic or non-metallic wire, embolic coil or a combination thereof. Non-limiting examples of coated, intravascular implants now follow.

The outside surface of a balloon catheter may be coated with the combination according to the present invention and could be released immediately or in a time dependent fashion. When the balloon expands and the wall of the vessel is in contact with the balloon, the release of the combination can begin. Small nanospheres of the agents can actually be transported into the vessel wall using the balloon so that these nanospheres ensure delivery over longer period of time.

The surface of a stent may be coated with the combination of agents and the stent is implanted inside the body. The stent struts could be loaded with several layers of the agents or with just a single layer. A transporter or a vehicle to load the agents on to the surface can also be applied to the stent. The graft material of the stent graft can also be coated (in addition to the stent or as an alternative) so that the material is transported intravascularly at the site of the location or the injury.

The drug delivery catheters that are used to inject drugs and other agents intravascularly can also be used to deliver the combination of agents. Other intravascular devices through which the transport can happen include atherectomy devices, filters, scaffolding devices, anastomotic clips, anastomotic bridges, suture materials etc.

The present invention envisions applying the coating directly to the intravascular implant. However, the coating can be applied to a primer, i.e. a layer or film of material upon which another coating is applied. Furthermore, the first and second agents can be incorporated in a polymer matrix. Polymeric matrices (bioabsorbable and biostable) can be used for delivery of the therapeutic agents. In some situations, when the agents are loaded on to the implant, there is a risk of quick erosion of the therapeutic agents either during the expansion process or during the phase during with the blood flow is at high shear rates at the time of implantation. In order to ensure that the therapeutic window of the agents is prolonged over extended periods of time, polymer matrices can be used.

These polymers could be any one of the following: semitelechelic polymers for drug delivery, thermo responsive polymeric micelles for targeted drug delivery, pH or temperature sensitive polymers for drug delivery, peptide and protein based drug delivery, water insoluble drug complex drug delivery matrices, polychelating amphiphilic polymers for drug delivery, bioconjugation of biodegradable poly lactic/glycolic acid for delivery, elastin mimetic protein networks for delivery, generically engineered protein domains for drug delivery, superporous hydrogel composites for drug delivery, interpenetrating polymeric networks for drug delivery, hyaluronic acid based delivery of drugs, photocrosslinked polyanhydrides with controlled hydrolytic delivery, cytokineinducing macromolecular glycolipids based delivery, cationic polysaccharides for topical delivery, n-halamine polymer coatings for drug delivery, dextran based coatings for drug delivery, fluorescent molecules for drug delivery, self-etching polymerization initiating primes for drug delivery, and bioactive composites based drug delivery.

Regardless of whether the coating includes a polymer matrix and where it is applied (directly on the implant, on top of a primer, or covered with a top coat), there are a number of different methods for applying the therapeutic coating according to the present invention. These include dip coating and spray coating. Applicant's co-pending U.S. patent application Ser. No. 10/320,795 filed Dec. 16, 2002 and U.S. Pat. No. 6,517,889 issued Feb. 11, 2003, both entitled “Process for Coating a Surface of a Stent”, discuss coating processes and disclose a novel method for coating a stent. The disclosures of these patent documents are incorporated herein by reference.

Another process for applying the therapeutic coating to an intravascular implant is as follows:

1. The implant is laser cut and then electropolished.

2. The electropolished implant is cleaned in a 1%-5% WN Potassium hydroxide or Sodium hydroxide for 1 hour. The temperature may be elevated to about 60 degrees Celsius to ensure proper cleaning. The cleaning can also be done with hexane or a solution of isopropyl alcohol.

3. The device is then washed with hot water. The washing may take place in a bath in which water is maintained at a constant temperature. Alternatively, the hot water is maintained on top of an ultrasonic bath so that the implant swirls as it is cleaned in the hot water.

4. The implant is dried at room temperature for up to 4 hours.

5. A primer is applied to the implant. The primer prepares the surface of the implant for the subsequent stages of bonding to the polymer.

6. Prepare functionalization chemicals. These chemicals could include hydride terminated polyphenyl_(dimethylhyrosiloxy) siloxanes; methylhydrosiloxane, phenylmethylsiloxane and methylhydrosiloxane-octylme-thylsiloxane copolymers, hydride terminated polydimethylsiloxanes, methylhydrosiloxanedimethylsiloxane copolymers; polymethylhyrosiloxanes, polyethylhydrosiloxanes. The chemicals could also include silanol functional siloxanes, like silanol terminated polydimethylsiloxanes; silanol terminated diphenylsiloxane-dimethylsiloxane copolymers; and silanol terminated polydiphenylsiloxanes. Suitable epoxy functional siloxanes include epoxy functional siloxanes include epoxypropoxypropyl terminated polydimethylsiloxanes and (epoxycyclohexylethyl) methylsiloxane-dimethylsiloxane copolymers.

7. The agents can be incorporated in the mixture of the polymer solution or can be bonded on to the surface of the polymer and also could be grafted on to the surface. One or more of the therapeutic agents is mixed with the coating polymers in a coating mixture. The therapeutic agent may be present as a liquid, a finely divided solid, or any other appropriate physical form. The mixture may include one or more additives, nontoxic auxiliary substances such as diluents, carriers, stabilizers etc. The best conditions are when the polymer and the drug have a common solvent. This provides a wet coating, which is a true solution.

8. The device is then placed in a mixture of functionalization chemicals for 2 hours at room temperature. An oscillating motion as described in the above-identified co-pending patent application can facilitate the coating process.

9. The device is then washed with methanol to remove any surface contaminants.

10. If there is a top coat of polymeric material that encapsulates the complete drug-polymer system, then the top coat is applied to the implant. The top coat can delay the release of the pharmaceutical agent, or it could be used as a matrix for the delivery of a different pharmaceutically active material.

11. The total thickness of the undercoat does not exceed 5 microns and the top coat is usually less than 2 microns.

In addition to applying a therapeutic coating to an intravascular implant, the implant can include an outer sheath where the sheath includes a therapeutic agent or agents to treat or prevent a disease process of a vascular disease of a patient. The intravascular implant may be, but is not limited to, a balloon catheter, stent, stent graft, drug delivery catheter, atherectomy device, filter, scaffolding device, anastomotic clip, anastomotic bridge, suture material, metallic or non-metallic wire, embolic coil or a combination thereof.

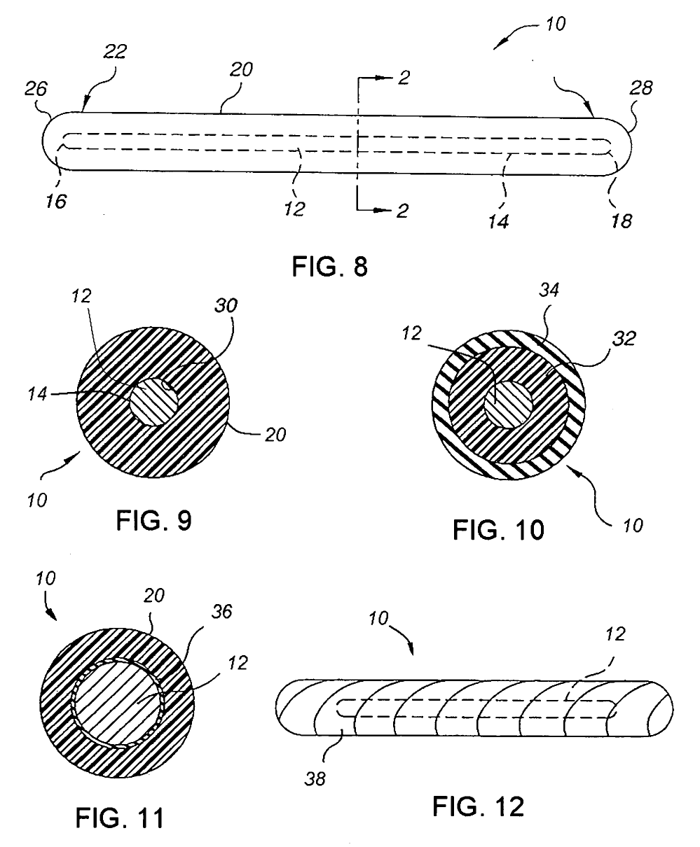

As seen in FIG. 8, a rationally designed therapeutically coated stent preform 10 includes an intravascular implant which takes the form of a wire or core 12 with a contact surface 14 and core ends 16 and 18. The core 12 of the stent preform 10 is preferably made of a rigid or rigidizable material. It may additionally be formed of a material that exhibits suitable ductility, with the material further being chosen based on its radiopacity in order to allow x-ray imaging. Various metals are appropriate for the substrate core, including but not limited to stainless steel, titanium, nickel, and combinations and alloys thereof. In particular, alloys that display the “shape memory” effect, such as a Ni-50% Ti alloy and several copper-base alloys, are appropriate. In a preferred embodiment, Nitinol is used for the core 12. As known to those skilled in the art, proper heat treatment of shape memory alloys allows structures to be created which assume several configurations depending on the temperature. Thus, a first shape can be used to facilitate implantation of the stent, and warming of the stent in the body lumen permits the stent to transform to a second shape that provides mechanical support to an artery. The second shape may be in the form of a coil to embolize a part of the anatomy or close a duct, or a mechanical scaffolding structure for vascular or nonvascular purposes. Also, cobalt-based alloys such as Eligiloy may be used as a metal core.

Other stiff materials can also be used to form the core 12, including carbon fibers, Kevlar, glass fibers, or the like. Some fiber filaments may not retain enough memory to maintain a preselected stent or coil shape. Thus, the stent may be fabricated by braiding several such filaments together to form a tubular structure. The filaments may be stretched to create a low profile, while releasing the filament from a stretched state allows it to assume a desirable shape. As is known to those skilled in the art, various braiding techniques may be employed, as well as various polymers or fillers. The core 12 is preferably substantially cylindrical in shape, although other core cross-sections may be used such as rectangular or hexagonal configurations.

As further seen in FIG. 8, the core 12 is surrounded by an outer sheath 20 having sheath ends 22 and 24 and caps 26 and 28. The sheath 20 includes a therapeutically effective amount of an agent or agents to treat a disease process in the pathology of a vascular disease. The agent or agents may include a macrolide immunosuppressant, anti-inflammatory agent, non-proliferative agent, anti-coagulant, anti-platelet agent, Tyrosine Kinase inhibitor, anti-infective agent, anti-tumor agent, anti-leukemic agent and a combination thereof. The sheath 20 may also serve as a sleeve or jacket which surrounds the core 12 to prevent the core from directly contacting a wall of a body lumen. The sheath 20 is preferably thin, and preferably an ultrathin tube of extruded polymer which may be microporous or macroporous. Although the sheath 20 may even have a thickness on the submicron level, in a preferred embodiment the sheath 20 has a thickness of between about 0.1 microns and 5 millimeters. The outer sheath 20 may be heat treated to ensure adhesion or bonding of the sheath 20 to the core 12. It may also be necessary to heat the composite to melt the polymer and permit it to flow, thereby not only allowing more effective bonding with the core 12 but also filling any gaps that may exist that expose the core 12.

Suitable polymers for this application include biocompatible polymers of particular permeability. The polymers can form a permeable, semi-permeable, or non-permeable membrane, and this property of the polymer may be selected during or after extrusion depending upon the particular polymer chosen. As shown in FIG. 9, the sheath 20 has an interior surface 30, which closely communicates with the contact surface 14 of the core 12. Numerous polymers are appropriate for use in stent preform 10, including but not limited to the polymers PTFE, ePTFE, PET, polyamide, PVC, PU, Nylon, hydrogels, silicone, silk, knitted or woven polyester fabric, or other thermosets or thermoplastics. In a preferred embodiment, the polymer is selected as a heat-shrinkable polymer. The sheath 20 may also be in the form of a thin film, which is deposited over the entire surface of core 12. A layer or multiple layers of submicron particles (nanoparticles) may also create a nanotube surrounding core 12. The sheath 20 must completely encapsulate core 12, and thus areas of the sheath form caps 26 and 28, as seen in FIG. 8.

The outer sheath 20 may be knitted or woven to form a braided configuration, however a sheath formed in this manner must still completely encapsulate the core 12. Sufficient tightness of the braiding around the core 12 is required, or alternatively the strands may be sealed together to form a continuous surface after braiding. The braided configuration is also designed to cover the ends 16 and 18 of core 12, as seen in FIG. 8.

FIG. 10 shows the outer sheath 20 formed of several layers of material. The layers may be of the same or varying thickness, and may be the same or different materials. In a preferred embodiment, a layer 32 is formed of a first polymer, and another layer 34 is a biological or other synthetic veneer which can preserve blood function. However, the biological material must be able to completely encapsulate the core 12, even after the core has been coiled or braided and formed into the shape of a stent. Thus, the biological coating should resist tearing and delamination which could result in exposure of core 12. If such a coating is applied prior to shaping the preform into a stent, it should be capable of withstanding the deformations and stresses that are induced by coil winding or braiding machines. It should also be capable of withstanding elevated temperatures if heat treatments are necessary.

The veneer may be an anticoagulent material such as heparin, coumadin, tichlopidiene, and chlopidogrel. The veneer may also be a genetic material such as angiogenic factors, tissue inhibiting material, growth factors such as VEGF, PDGF, and PGF, as well as thrombin inhibiting factors. The growth factors and angiogenic factors can be sourced biologically, for example through porcine, bovine, or recombinant means, and the growth factors even can be derived from the patient's own body by processing blood from the patient. The veneer may be applied to the polymer layer by dipping the outer sheath 20 into growth factors for several minutes to promote attachment, and additional factors may be added to help effectuate the attachment. The growth factors can further be encapsulated in a release mechanism made of liposomes, PLA, PGA, HA, or other release polymers. Alternatively, the growth factors can be encapsulated in non-controlled release, naturally-derived polymers such as chitosan and alginate.

In an alternate embodiment, the veneer can be sandwiched between the micropores of the polymer layer so that a controlled release occurs. In yet another alternate embodiment, a multilayer outer sheath 20 can be formed wherein an active release substrate polymer is attached to a layer of a different polymer, or sandwiched between two layers of either the same or different polymers. The outer sheath 20 may otherwise be formed of an inert polymer, or of an inert polymer surrounding an active polymer.

FIG. 11 shows another embodiment of the stent preform 10 according to the present invention. The stent preform 10 includes a core 12, an outer sheath 20, and a lubricious lining 36. The lubricious lining 36 is disposed between core 12 and outer sheath 20 to facilitate insertion of core 12 into the sheath 20. The lubricious lining 36 may be attached to core 12 or outer sheath 20, or it may be separate. The lining 36 permits a tight fit between core 12 and outer sheath 20 by providing a lubricated surface on which either can be slid relative to the other, thereby allowing the inner dimension of the outer sheath 20 to very closely match the outer dimension of the core 12.

In addition to applying a therapeutic coating or sheath to an intravascular implant, the implant can include therapeutic tape where the tape includes a therapeutic agent or agents to treat or prevent a disease process in the pathology of a vascular disease. The agent or agents may include a macrolide immunosuppressant, anti-inflammatory agent, non-proliferative agent, anti-coagulant, anti-platelet agent, Tyrosine Kinase inhibitor, anti-infective agent, anti-tumor agent, anti-leukemic agent and a combination thereof. The intravascular implant may be, but is not limited to, a balloon catheter, stent, stent graft, drug delivery catheter, atherectomy device, filter, scaffolding device, anastomotic clip, anastomotic bridge, suture material, metallic or non-metallic wire, embolic coil or a combination thereof.

For example, FIG. 12 shows a stent preform 10 with the core 12 wrapped in tape 38. The tape 38 completely covers core 12 so that the core is isolated from the lumen walls after implantation. In an alternate embodiment, the tape 38 is applied around a core that is already covered with another coating or layer of polymer. The tape 38 may be applied to the core using a winding machine or other suitable means.

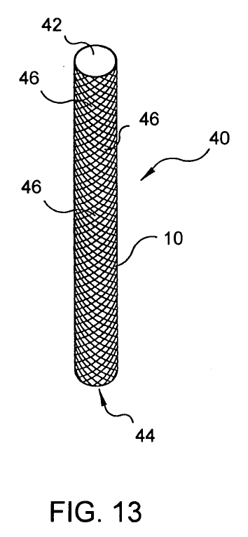

FIG. 13 shows a braided stent 40 made from a stent preform 10. In an alternative embodiment of braided stent 40, multiple stent preforms 10 may be used. The ends 42 and 44 may be pulled to extend the braided stent 40 to a longer length, thereby also decreasing the inner diameter of the stent. When released, the stent returns to a relaxed length and diameter. Open areas 46 between the stent preform walls permit new tissue growth which may eventually cover the stent structure. The braided stent, or other shapes or coils forming a stent, can be mounted on top of an expansile device such as a balloon catheter, which expands the stent from a relaxed diameter to an elongated diameter. A stent 40 formed from at least one rationally designed, therapeutically coated stent preform 10 can prevent or treat a disease process or processes in the pathology of a vascular disease. The therapeutic agent or agents of the stent preform 10 may include a macrolide immunosuppressant, anti-inflammatory agent, non-proliferative agent, anti-coagulant, anti-platelet agent, Tyrosine Kinase inhibitor, anti-infective agent, anti-tumor agent, anti-leukemic agent and a combination thereof.

A delivery housing in combination with a shaft may be used to insert the stent into a lumen. The housing may have a cylindrical shape, and the stent, loaded on the shaft in extended state, is placed in the housing. Once the housing is inserted into the lumen, the stent may be slowly withdrawn from the housing while supported and guided by the shaft, and allowed to return to its unextended shape having a greater diameter. The housing and shaft are completely withdrawn from the lumen leaving the stent as a lining inside the vessel wall to exclude blockage from the vessel. By emobilizing the duct with a stent having an isolated core, the stent is more readily accepted by the body. This implantation method can be applied to any anatomical conduit.

Stents incorporating shape memory materials may be heat treated in various states to permit the stretched configuration. Although the core may require treatment at 650 degrees Celsius, care must be exercised when fabricating the stents of the present invention since a Polymer overlayer will be provided.

Stent preforms may be spooled to permit storage in a roll form, or may also be kept in an unrolled state.

U.S. Pat. No. 6,475,235 issued on Nov. 5, 2002 and entitled, “Encapsulated Stent Preform” further discusses an outer sheath and tape for covering an implant, and more particularly, discusses a stent preform. Also, U.S. patent application Ser. No. 10/286,805 filed Nov. 4, 2002 discloses a stent formed from encapsulated stent preforms. The disclosures of those patent documents are incorporated herein by reference.

While various descriptions of the present invention are described above, it should be understood that the various features could be used singularly or in any combination thereof. Therefore, this invention is not to be limited to only the specifically preferred embodiments Depicted herein.

Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A method for treating a vascular disease of a patient with an intravascular implant, the method comprising:
identifying a disease process in the pathology of the vascular disease;
selecting a first agent to treat or prevent the vascular disease;
coating at least a portion of the intravascular implant with a therapeutically effective amount of the first agent; and
implanting the intravascular implant in the patient.

2. A method as defined in claim 1 wherein the disease process is identified using a technique selected from the group consisting of an angiogram, fluoroscopy, CT scan, MRI, intravascular MRI, lesion temperature determination, genetic determination and a combination thereof.

3. A method as defined in claim 2 wherein the disease process is selected from the group consisting of acute myocardial infarction, thrombotic lesions, unstable angina, fibrotic disease, total occlusion, hyperproliferative vascular disease, vulnerable plaque, diabetic vascular diffused disease and a combination thereof.

4. A method as defined in claim 3 wherein the first agent acts on a calcium independent cellular pathway.

5. A method as defined in claim 4 wherein the first agent is a macrolide immunosuppressant.

6. A method as defined in claim 5 further including selecting a second agent to treat or prevent the vascular disease, and wherein coating at least a portion of the intravascular implant with a therapeutically effective amount of the first agent includes coating at least a portion of the intravascular implant with a therapeutically effective amount of the second agent.

7. A method as defined in claim 6 wherein the second agent is selected from the group consisting of an anti-inflammatory agent, non-proliferative agent, anti-coagulant, anti-platelet agent, Tyrosine Kinase inhibitor, anti-infective agent, anti-tumor agent, anti-leukemic agent and a combination thereof.

8. A method as defined in claim 1 wherein coating at least a portion of the intravascular implant includes coating at least a portion of the intravascular implant with a polymer matrix.

9. A method as defined in claim 8 wherein the polymer matrix includes a bioabsorbable polymer.

10. A method as defined in claim 9 wherein the bioabsorbable polymer is selected from the group consisting of poly-α hydroxy acids, polyglycols, polytyrosine carbonates, starch, gelatins, cellulose and combinations thereof.

11. A method as defined in claim 10 wherein the therapeutically effective amount of the first agent is dispersed within the bioabsorbable polymer.

12. A method as defined in claim 1 wherein the intravascular implant is selected from the group consisting of a balloon catheter, stent, stent graft, drug delivery catheter, atherectomy device, filter, scaffolding device, anastomotic clip, anastomotic bridge, suture material, wire, embolic coil and a combination thereof.

13. A method as defined in claim 12 wherein the intravascular implant includes a primer layer upon which the coating is applied.

14. A method as defined in claim 13 wherein the primer layer is made of a bioabsorbable polymer.

15. A method as defined in claim 13 wherein the primer layer is made of a biostable polymer.

16. A method as defined in claim 13 further including a top coat applied over the coating.

17. A method of making a therapeutically coated intravascular implant for treating a vascular disease, the method comprising:
identifying a disease process in the pathology of a vascular disease of a patient;
selecting a first agent to treat or prevent the vascular disease; and
coating at least a portion of the intravascular implant with a therapeutically effective amount of the first agent.

18. A method as defined in claim 17 wherein the disease process is identified using a technique selected from the group consisting of an angiogram, fluoroscopy, CT scan, MRI, intravascular MRI, lesion temperature determination, genetic determination and a combination thereof.

19. A method as defined in claim 18 wherein the disease process is selected from the group consisting of acute myocardial infarction, thrombotic lesions, unstable angina, fibrotic disease, total occlusion, hyperproliferative vascular disease, vulnerable plaque, diabetic vascular diffused disease and a combination thereof.

20. A method as defined in claim 19 further including selecting a second agent to treat or prevent the vascular disease, and wherein coating at least a portion of the intravascular implant with a therapeutically effective amount of the first agent includes coating at least a portion of the intravascular implant with a therapeutically effective amount of the second agent.

21. A method as defined in claim 20 wherein the first agent is rapamycin.

22. A method as defined in claim 21 wherein the second agent is selected from the group consisting of an anti-inflammatory agent, non-proliferative agent, anti-coagulant, anti-platelet agent, Tyrosine Kinase inhibitor, anti-infective agent, anti-tumor agent, anti-leukemic agent and a combination thereof.

23. A therapeutic intravascular implant coating for coating at least a portion of an intravascular implant to treat or prevent a vascular disease of a patient, the coating comprising:
a therapeutically effective amount of rapamycin; and
a therapeutically effective amount of a second agent,
wherein the second agent is selected based on the vascular disease of the patient.

24. A coating as defined in claim 23 wherein the second agent is selected from the group consisting of an anti-inflammatory agent, non-proliferative agent, anti-coagulant, anti-platelet agent, Tyrosine Kinase inhibitor, anti-infective agent, anti-tumor agent, anti-leukemic agent and a combination thereof.

25. A coating as defined in claim 24 wherein the intravascular implant is selected from the group consisting of a balloon catheter, stent, stent graft, drug delivery catheter, atherectomy device, filter, scaffolding device, anastomotic clip, anastomotic bridge, suture material, wire, embolic coil and a combination thereof.
